(19) United States
(12) Patent Application Publication
(10) Pub. No.: US 2012/0022309 A1
(43) Pub. Date: Jan. 26, 2012

(54) METHOD FOR PRODUCING AROMATIC HYDROCARBON

(75) Inventors: Hongtao Ma, Tokyo (JP); Yuji Ogawa, Saitama (JP)

(73) Assignee: MEIDENSHA CORPORATION

(21) Appl. No.: 13/146,274

(22) PCT Filed: Jan. 15, 2010
(86) PCT No.: PCT/JP2010/050378

§ 371 Date: Jul. 26, 2011

(30) Foreign Application Priority Data
Feb. 12, 2009 (JP) 2009-029703

(51) Int. Cl. C07C 2/76

(52) U.S. Cl. 585/415

(57) ABSTRACT

[Object] To produce aromatic hydrocarbon stably for a long time maintaining a high aromatic hydrocarbon yield when the aromatic hydrocarbon is produced upon making a contact reaction between lower hydrocarbon and a catalyst.

[Solving Means] In a method of producing aromatic hydrocarbon, including repeating a reaction step for obtaining aromatic hydrocarbon upon making a contact reaction between lower hydrocarbon and a catalyst and a regeneration step for regenerating the catalyst used in the reaction step, carbon dioxide in an amount of 0.33 to 1.6% by volume relative to an amount of the lower hydrocarbon is added to the lower hydrocarbon, in the reaction step.

### TECHNICAL FIELD

This invention relates to a method of effectively producing aromatic compound such as benzene or the like and hydrogen from lower hydrocarbon such as methane, ethane, propane and/or the like, and particularly to the method of effectively producing aromatic compound such as benzene or the like from lower hydrocarbon by using a catalyst.

### BACKGROUND ART

Hitherto aromatic compound such as benzene, toluene, xylene or the like is produced mainly from naphtha. As production methods for naphthalenes, a non-catalyst method such as a solvent extraction process for coal or the like and a gas thermal cracking process for natural gas, acetylene or the like are employed.

However, according to these conventional methods, only several percents of benzene or naphthalenes can be obtained relative to a raw material such as coal, acetylene or the like. Additionally, much by-product aromatic compounds and hydrocarbons, tar and insoluble carbon residues are produced, which offers problems. Additionally, there is such a drawback that the solvent extraction process for coal or the like requires much organic solvent.

As a production process for hydrogen gas, a steam reforming process for natural gas or naphtha is the mainstream; however, this requires a high temperature such as around 900° C., requires burning much raw material in order to maintain a reforming temperature, and uses steam in the amount of 3 to 4 times the theoretical required amount in order to prevent the activity lowering of a catalyst thus consuming much energies. Further, there is, for example, such a problem that much carbon dioxide as a grovel warming substance is generated as reforming and burning products.

As a method for producing aromatic hydrocarbon such as benzene, naphthalene or the like from lower hydrocarbon such as particularly methane, it is known that methane is directly decomposed on a catalyst in a system in which no oxygen exists, which is a direct convention process for methane (see, for example, Patent Citations 1 to 4). As the catalyst in this case, molybdenum, rhenium or the like carried on HZSM-5 zeolite is assumed to be effective (see, for example, Non-patent Citations 1 and 2). Even in case that such a catalyst is used, there are such problems to be solved that a catalytic activity is remarkably lowered owing to carbon deposition, and a methane conversion rate is low.

As a measure for solving the above problems, Patent Citation 1 discloses a method for producing aromatic compound and hydrogen from lower hydrocarbon such as methane, ethane or the like, in which CO₂ or CO is added to a gas to be reacted thereby suppressing carbon deposition as a side reaction thus reducing a catalytic activity lowering due to a reaction. Additionally, Patent Citations 2 to 4 disclose that a contact reaction for lower hydrocarbon and a catalyst regeneration reaction are alternately repeated thereby stably producing aromatic hydrocarbon and hydrogen.

### PRIOR ART CITATION

### Patent Citation

Patent Citation 1: Japanese Patent No. 3745885

Patent Citation 2: Japanese Patent No. 3985038

Patent Citation 3: Japanese Patent Provisional Publication No. 2008-266244

Patent Citation 4: Japanese Patent Provisional Publication No. 2008-302291

### Non-Patent Citation

Non-patent Citation 1: JOURNAL OF CATALYSIS, 1999, Volume 182, p. 92-103

Non-patent Citation 2: JOURNAL OF CATALYSIS, 2000, Volume 190, p. 276-283

### DISCLOSURE OF THE INVENTION

### Problems to be Solved by the Invention

According to Patent Citation 1, addition of high concentration of CO2 or CO is required in order to carry out a reaction for obtaining aromatic hydrocarbon upon bringing methane into contact with a catalyst, stably for a long time.

However, if excessive CO₂ or CO is added, an aromatization reaction is obstructed thereby providing such a problem that the yield of aromatic hydrocarbon is lowered.

In case that CO₂ or CO is not added, a very high benzene yield is obtained at an initial period; however, a carbon deposition reaction heavily occurs so that the activity of a catalyst is lost for a short period of time.

Coke deposited on a catalyst in a reaction step can be removed in a regeneration step; however, removal-difficult coke which is difficult to be removed in the relatively short time regeneration step is produced according to a reaction time. In case that this removal-difficult coke is accumulated, the benzene yield cannot be restored to its initial level so as to be gradually reduced even if the reaction step and the regeneration step are repeated. In order to remove such a removal-difficult coke, the regeneration step for a ling time is required as disclosed, for example, in Patent Citation 4.

Accordingly, an object of the present invention is to provide a method for producing aromatic hydrocarbon upon making a contact reaction between lower hydrocarbon and a catalyst, in which the yield of aromatic hydrocarbon is maintained high and is not lowered even upon repetition of a reaction step and a regeneration step.

### Means for Solving the Problems

A method of producing aromatic hydrocarbon, according to the present invention, to attain the above object comprises repeating a reaction step for obtaining aromatic hydrocarbon upon making a contact reaction between lower hydrocarbon and a catalyst and a regeneration step for regenerating the catalyst used in the reaction step, which is characterized in that carbon dioxide in an amount of 0.33 to 1.6% by volume relative to an amount of the lower hydrocarbon is added to the lower hydrocarbon, in the reaction step.

The reaction step may be changed over into the regeneration step, based on a yield of benzene produced in the reaction step. Additionally, a reaction time in the reaction step may be within 5 hours.

The regeneration step may be carried out by bringing the catalyst into contact with hydrogen.

### Effects of the Invention

According to the above invention, when aromatic hydrocarbon is produced by reacting lower hydrocarbon and the catalyst, aromatic hydrocarbon can be produced stably for a long time while maintaining a high aromatic hydrocarbon yield.

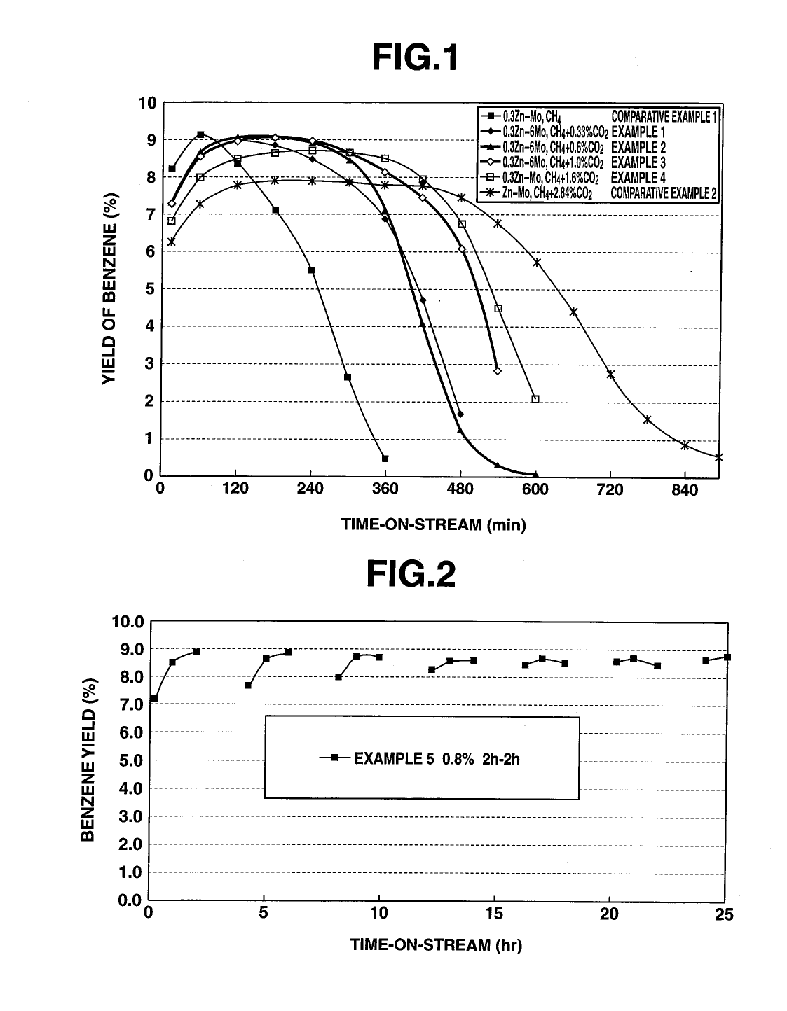

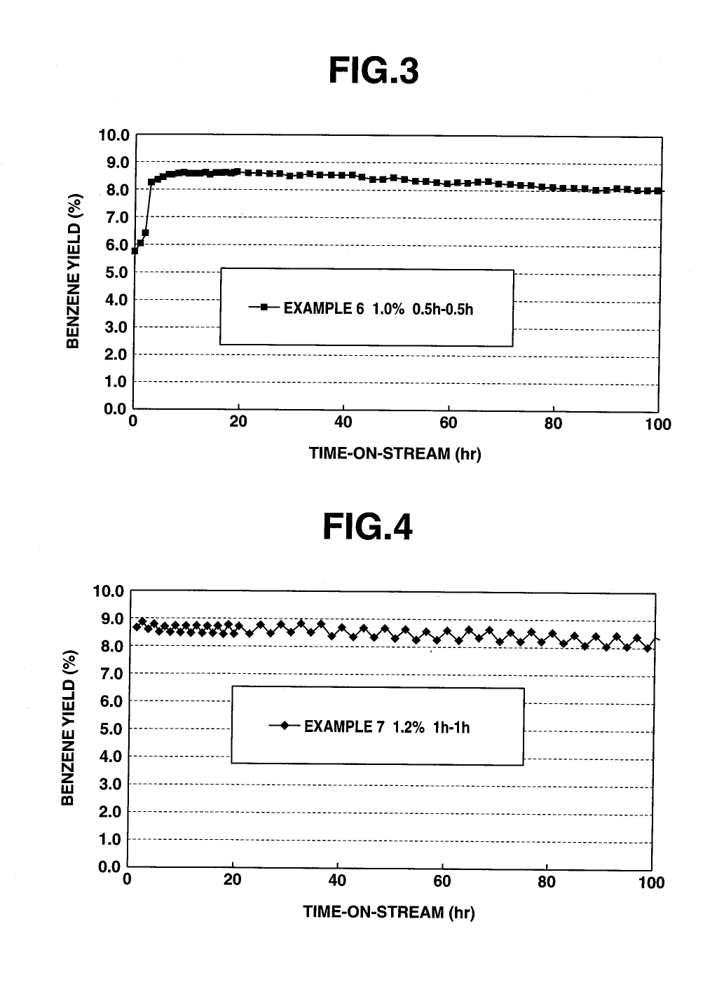

### BRIEF DESCRIPTION OF THE DRAWINGS

[FIG. 1] is a graph showing a time-based change of a benzene yield in case that a catalytic reaction is continuously made;

[FIG. 2] is a graph showing a time-based change of a benzene yield in case that a catalytic reaction step and a regeneration step are repeated under a condition in connection with Example 5;

[FIG. 3] is a graph showing a time-based change of a benzene yield in case that a catalytic reaction step and a regeneration step are repeated under a condition in connection with Example 6; and

[FIG. 4] is a graph showing a time-based change of a benzene yield in case that a catalytic reaction step and a regeneration step are repeated under a condition in connection with Example 7.

### MODE FOR CARRYING OUT THE INVENTION

This invention relates to a method of producing aromatic hydrocarbon upon making a reaction of lower hydrocarbon in presence of a catalyst, which is characterized by adding carbon dioxide in an amount smaller than an excessive amount during the reaction and by regenerating the catalyst by substituting a reaction gas with a regeneration gas every a certain time. A remarkable carbon (coke) deposition can be suppressed by adding carbon dioxide in the amount smaller than the excessive amount, while the reaction can be made for a long time maintaining a high yield without accumulation of removal-difficult coke by making a catalytic reaction upon substituting the reaction gas with the regeneration gas every a certain time.

Examples of a reactor to be used in the method of producing aromatic hydrocarbon according to the present invention are a fixed bed reactor, a fluidized bed reactor and the like.

A temperature of the reaction is 600° C. to 900° C., preferably 700° C. to 850° C. and more preferably 750° C. to 830° C.

A pressure of the reaction is 0.1 MPa to 0.9 MPa, preferably 0.1 MPa to 0.5 MPa.

A charging amount of a raw material is represented as a weight hourly space velocity (WHSV) relative to an amount of a catalyst, and is 150 to 70000 [ml/g-MFI/h], preferably 500 to 30000 [ml/g-MFI/h] and more preferably 1400 to 14000 [ml/g-MFI/h].

A zeolite-based catalyst is not particularly limited if it is a zeolite catalyst having a catalytic activity. As the zeolite-based catalyst, for example, zeolite-based catalysts such as mordenite; erionite; ferrierite; “ZSM-5”, “ZSM-4”, “ZSM-8”, “ZSM-11”, “ZSM-12”, “ZSM-20”, “ZSM-40”, “ZSM-35” and/or “ZSM-48” commercially available from Mobil Oil Corporation; and/or the like can be used. Additionally, as the zeolite-based catalyst, known zeolite-based catalysts, for example, crystalline aluminosilicate including so-called meso-porous zeolite and/or the like such as “MCM-41”, “MCM-48”, “MCM-50”, “FSM-41”,“M41S” and/or the like; different element-containing zeolite such as porosilicate, gallosilicate, ferroaluminosilicate, titanosilicate, and/or the like; and/or the like can be used. Of these zeolite-based catalysts, those suitable for a hydration reaction for olefin are crystalline aluminosilicate and gallosilicate having a pentasil structure.

As the zeolite-based catalyst, that of the proton exchange type (H-type) is usually used. Additionally, a part of protons in the zeolite-based catalyst may be substituted with at least one kind selected from alkali metal such as Na, K, Li and the like, alkaline earth element such as Mg, Ca, Sr and the like, transition metal element such as Fe, Co, Ni, Ru, Pd, Pt, Zr, Ti and the like. Additionally, the zeolite-based catalyst may contain Ti, Zr, Hf, Cr, Mo, W, Th, Cu, Ag and/or the like in a suitable amount.

The zeolite-based catalyst is not particularly restricted in its state and may be used in any state such as powdery state, granular state and the like. Additionally, alumina, titania, silica, clayey compound and/or the like may be used as a carrier or a binder for the zeolite-based catalyst.

The zeolite-based catalyst may be used upon being formed into pellet or extruded product by adding a binder such as silica, alumina, clay and/or the like to it.

In the present invention, lower hydrocarbon means a mixture containing methane in an amount of at least 50% and preferably not less than 70% and additionally containing saturated and unsaturated hydrocarbons having a carbon number of 2 to 6. Examples of these saturated and unsaturated hydrocarbons having a carbon number of 2 to 6 are ethane, propane, propylene, n-butane, isobutane, n-butene, isobutene and the like.

Hereafter, the present invention will be discussed further in detail with reference to Examples.

### EXAMPLES

A lower hydrocarbon aromatization catalyst (hereafter referred to as catalyst) was prepared by a preparation method as discussed below, using H-type ZSM-5 zeolite (SiO₂/Al₂O₃=40) as a metallosilicate carrier.

400 g of HZSM-5 subjected to a silane treatment was dissolved in an aqueous solution prepared by dissolving certain amounts of ammonium molybdate and zinc nitrate into 2000 ml of ion-exchanged water, and stirring was made at room temperature for 3 hours so that zinc and molybdenum were carried on HZSM-5. Zinc and molybdenum were carried on HZSM-5 in a mol ratio of 0.3:1.

After the obtained ZSM carrying zinc and molybdenum (Zn(1.23 wt %)/Mo (6 wt %)/HZSM-5) was dried, it was calcined at 550° C. for 8 hours thereby obtaining catalyst powder. Further, an inorganic binder was added to this catalyst powder, and then an extrusion formation into the pellet state was made, followed by calcination, thereby producing a catalyst.

Using the obtained catalyst, a test for producing aromatic hydrocarbon from lower hydrocarbon was conducted. Evaluation of the catalyst was accomplished with the yield of benzene relative to lower hydrocarbon which was flown through. The yield of benzene was defined as indicated below.

Benzene yield (%)={(quantity of benzene produced (mol))/(quantity of methane supplied for a methane reforming reaction(mol))}×100

Hereinafter, common reaction conditions in each test are shown below.
Reaction temperature: 780° C.;
Pressure: 0.15 MPa; and
Weight hourly space velocity (WHSV): 30000 ml/g-MFI/h

A pretreatment for the catalyst was conducted as follows: The temperature of the catalyst was raised to 550° C. in the stream of air and kept for 2 hours. Thereafter, air was substituted with a pretreatment gas containing 20% of methane and 80% of hydrogen, and the temperature of the catalyst was raised to 700° C. and kept for 3 hours. Thereafter, the pretreatment gas was substituted with a reaction gas, and the temperature of the catalyst was raised to a certain temperature (780° C.) thus accomplishing the evaluation of the catalyst.

Hydrogen, argon and methane were analyzed by TCD-GC, while aromatic hydrocarbons such as benzene, toluene, xylene and naphthalene and the like were analyzed by FID-GC.

FIG. 1 is a graph showing a time-based change of the benzene yield upon continuously carrying out a catalytic reaction, in case that the condition of the reaction gas was changed as in Comparative Examples 1 and 2, and Examples 1 to 4.

In Comparative Example 1, a reaction was carried out for the above-mentioned reaction gas formed by adding no carbon dioxide to 100 parts (volume %) of methane during the reaction, in which a lapsed time-based observation of the result of the above-mentioned analysis was made.

In Example 1, a reaction was carried out for the above-mentioned reaction gas formed by adding 0.33 part (volume %) of carbon dioxide to 100 parts (volume %) of methane during the reaction, in which a lapsed time-based observation of the result of the above-mentioned analysis was made.

In Example 2, a reaction was carried out for the above-mentioned reaction gas formed by adding 0.6 part (volume %) of carbon dioxide to 100 parts (volume %) of methane during the reaction, in which a lapsed time-based observation of the result of the above-mentioned analysis was made.

In Example 3, a reaction was carried out for the above-mentioned reaction gas formed by adding 1.0 part (volume %) of carbon dioxide to 100 part (volume %) of methane during the reaction, in which a lapsed time-based observation of the result of the above-mentioned analysis was made.

In Example 4, a reaction was carried out for the above-mentioned reaction gas formed by adding 1.6 part (volume %) of carbon dioxide to 100 parts (volume %) of methane during the reaction, in which a lapsed time-based observation of the result of the above-mentioned analysis was made.

In Comparative Example 2, a reaction was carried out for the above-mentioned reaction gas formed by adding 2.84 parts (volume %) of carbon dioxide to 100 parts (volume %) of methane during the reaction, in which a lapsed time-based observation of the result of the above-mentioned analysis was made.

As apparent from FIG. 1, in Comparative Example 1, the benzene yield is high. However, lowering in benzene yield along with lapse of the reaction time is remarkable.

In Examples 1 to 4, the benzene yield is not different from that in Comparative Example 1, in which a catalyst stability is improved as the addition amount of carbon dioxide increases. A remarkable effect can be exhibited particularly in case that the addition amount of carbon dioxide is 0.6 volume % (Example 2) and in case that the addition amount of carbon dioxide is 1.0 volume % (Example 3). Since the reaction time for maintaining a high benzene yield is about 5 hours, it is confirmed to be effective that the aromatic hydrocarbon production reaction and the catalyst regeneration reaction are repeated within this reaction time range.

As apparent from Comparative Example 2, as the addition amount of carbon dioxide increases, the stability of the catalyst is improved; however, the benzene yield lowers. This is assumed to result from the fact that an aromatization reaction is suppressed with excessive carbon dioxide.

FIGS. 2 to 4 are graphs showing time-based changes of the benzene yields in case of repeating the catalyst reaction step and the regeneration step respectively under conditions in connection with Examples 5 to 7. The respective conditions are shown below.

In Example 5, a reaction for 2 hours was carried out for the above-mentioned reaction gas formed by adding 0.8 part (volume %) of carbon dioxide to 100 parts (volume %) of methane during the reaction. Thereafter, the reaction gas was substituted with hydrogen gas to carry out a regeneration for 2 hours. These reaction and regeneration were alternately changed thereby continuously producing aromatic hydrocarbon, in which a lapsed time-based observation of the result of the above-mentioned analysis was made.

In Example 6, a reaction for 0. 5 hour was carried out for the above-mentioned reaction gas formed by adding 1.0 part (volume %) of carbon dioxide to 100 parts (volume %) of methane during the reaction. Thereafter, the reaction gas was substituted with hydrogen gas to carry out a regeneration for 0.5 hour. These reaction and regeneration were alternately changed thereby continuously producing aromatic hydrocarbon, in which a lapsed time-based observation of the result of the above-mentioned analysis was made.

In Example 7, a reaction for 1 hour was carried out for the above-mentioned reaction gas formed by adding 1.2 part (volume %) of carbon dioxide to 100 parts (volume %) of methane during the reaction. Thereafter, the reaction gas was substituted with hydrogen gas to carry out a regeneration for 1 hour. These reaction and regeneration were alternately changed thereby continuously producing aromatic hydrocarbon, in which a lapsed time-based observation of the result of the above-mentioned analysis was made.

As shown in FIGS. 2 to 4, it will be understood that an aromatic hydrocarbon production reaction can be repeatedly and continuously accomplished many times maintaining a high benzene yield by adding 0.8 to 1.2 parts (volume %) of carbon dioxide as the reaction gas to 100 parts (volume %) of methane. In other words, it will be understood that aromatic hydrocarbon can be produced without being affected by carbon deposition even upon repeating a cycle of the reaction and the regeneration.

Additionally, it is preferable that a changeover time of the reaction and the regeneration is within a time for which the yield is the most stable (within about 5 hours in Examples 2 and 3 in FIG. 1). It will be understood that the regeneration of the catalyst is possible regardless of the span of the changeover time if the catalytic reaction is made upon being changed over within 2 hours.

As discussed above, according to the method of producing aromatic hydrocarbon by making a contact reaction of lower hydrocarbon with the catalyst in the present invention, the removal-difficult coke can be prevented from accumulating, so that it is possible to make a production reaction for a long time maintaining a high benzene yield (catalytic activity).

Accordingly, it becomes possible to produce aromatic hydrocarbon without frequently changing the catalytic reaction step and the catalyst regeneration step. Further, the benzene yield cannot be lowered though the catalytic reaction step and the catalyst regeneration step are repeated.

Additionally, the amount of carbon dioxide to be added in the reaction gas is 0.33 to 1.6 parts (volume %), preferably 0.6 to 1.2 parts and more preferably 0.8 to 1.2 parts (volume %) of carbon dioxide relative to 100 parts (volume %) of methane.

The present invention is not limited to the Examples, so that reaction conditions, catalysts (kinds and amounts of metals carried thereon), and the like may be suitably selectable.

1. A method of producing aromatic hydrocarbon, comprising:
repeating a reaction step for obtaining aromatic hydrocarbon upon making a contact reaction between lower hydrocarbon and a catalyst and a regeneration step for regenerating the catalyst used in the reaction step,
characterized in that carbon dioxide in an amount of 0.33 to 1.2% by volume relative to an amount of the lower hydrocarbon is added to the lower hydrocarbon, in the reaction step.

2. The method of producing aromatic hydrocarbon as claimed in claim 1, characterized in that the reaction step is changed over into the regeneration step, based on a yield of benzene produced in the reaction step.

3. The method of producing aromatic hydrocarbon as claimed in claim 2, characterized in that a reaction time in the reaction step is within 5 hours.

4. The method of producing aromatic hydrocarbon as claimed in claim 1, characterized in that the regeneration step is carried out by bringing the catalyst into contact with hydrogen.

5. The method of producing aromatic hydrocarbon as claimed in claim 2, characterized in that the regeneration step is carried out by bringing the catalyst into contact with hydrogen.

6. The method of producing aromatic hydrocarbon as claimed in claim 3, characterized in that the regeneration step is carried out by bringing the catalyst into contact with hydrogen.
